(19) United States
(12) Patent Application Publication
(10) Pub. No.: US 2023/0076903 A1
(43) Pub. Date: Mar. 9, 2023

(54) CUE-BASED MEDICAL REPORTING ASSISTANCE

(71) Applicant: Siemens Healthcare GmbH, Erlangen (DE)

(72) Inventor: Sailesh CONJETI, Erlangen (DE)

(73) Assignee: Siemens Healthcare GmbH, Erlangen (DE)

(21) Appl. No.: 17/894,272

(22) Filed: Aug. 24, 2022

(30) Foreign Application Priority Data
Aug. 27, 2021 (EP) 21193589.5

(51) Int. Cl. G16H 30/20; G16H 10/60

(52) U.S. Cl. CPC G16H 30/20; G16H 10/60; G16H 30/40

(57) ABSTRACT

A computer-implemented method comprises: obtaining a medical imaging dataset of a current examination of a patient; determining, based on the medical imaging dataset, one or more diagnostic cues using a processing algorithm, wherein the one or more diagnostic cues are associated with patient-specific diagnostic findings; and controlling a user interface to pose the one or more diagnostic cues to a user, as part of a workflow for drawing up a current medical report for the current examination.

### CROSS-REFERENCE TO RELATED APPLICATION(S)

The present application claims priority under 35 U.S.C. § 119 to European Patent Application No. 21193589.5, filed Aug. 27, 2021, the entire contents of which are incorporated herein by reference.

### FIELD

Various examples of the disclosure pertain to a workflow for drawing up a medical report. Various examples of the disclosure specifically relate to one or more diagnostic cues being posed to the user.

### BACKGROUND

Medical practitioners are trained in drawing up medical reports for patients based on a diagnosis. Typically, the diagnosis is based on medical imaging datasets, obtained by medical measurements such as tomography.

### SUMMARY

The inventors have discovered there is a need for advanced techniques of assisting medical practitioners in drawing up medical reports.

This need is met by one or more example embodiments of the disclosure.

Techniques are disclosed that pose one or more diagnostic cues to a user as part of a workflow for drawing up a medical report. The one or more diagnostic cues can assist the user, e.g., a medical practitioner, in drawing up the medical report.

A computer-implemented method is provided. The method includes obtaining medical imaging dataset of a current examination of a patient. The method further includes, based on the medical imaging dataset, determining one or more diagnostic cues using a processing algorithm, the one or more diagnostic cues being associated with patient-specific diagnostic findings. The method further includes, as part of a workflow for drawing up a current medical report for the current examination, controlling a user interface to pose the one or more diagnostic cues to a user.

A computer program or a computer program product or a computer-readable storage medium includes program code. The program code can be loaded an executed by at least one processor. Upon executing the program code, the at least one processor performs a method. The method includes obtaining medical imaging dataset of a current examination of a patient. The method further includes, based on the medical imaging dataset, determining one or more diagnostic cues using a processing algorithm, the one or more diagnostic cues being associated with patient-specific diagnostic findings. The method further includes, as part of a workflow for drawing up a current medical report for the current examination, controlling a user interface to pose the one or more diagnostic cues to a user.

A device includes a processer. The processor is configured to obtain medical imaging dataset of a current examination of a patient. The processor is configured to determine one or more diagnostic cues based on the medical imaging dataset and using a processing algorithm, the one or more diagnostic cues being associated with patient-specific diagnostic findings. The processor is also configured to, as part of a workflow for drawing up a current medical report for the current examination, control a user interface to pose the one or more diagnostic cues to a user.

It is to be understood that the features mentioned above and those yet to be explained below may be used not only in the respective combinations indicated, but also in other combinations or in isolation without departing from the scope of the present invention.

### BRIEF DESCRIPTION OF THE DRAWINGS

FIG. 1 schematically illustrates a data processing for drawing up a medical report according to various examples.

FIG. 2 schematically illustrates a device according to various examples.

FIG. 3 is a flowchart according to various examples.

FIG. 4 schematically illustrates a workflow according to various examples.

FIG. 5 is a flowchart of a method according to various examples.

FIG. 6 schematically illustrates a processing algorithm for determining one or more diagnostic cues according to various examples.

FIG. 7 schematically illustrates a processing algorithm for determining one or more diagnostic cues according to various examples.

FIG. 8 schematically illustrates a processing algorithm for determining one or more diagnostic cues according to various examples.

### DETAILED DESCRIPTION

Some examples of the present disclosure generally provide for a plurality of circuits or other electrical devices. All references to the circuits and other electrical devices and the functionality provided by each are not intended to be limited to encompassing only what is illustrated and described herein. While particular labels may be assigned to the various circuits or other electrical devices disclosed, such labels are not intended to limit the scope of operation for the circuits and the other electrical devices. Such circuits and other electrical devices may be combined with each other and/or separated in any manner based on the particular type of electrical implementation that is desired. It is recognized that any circuit or other electrical device disclosed herein may include any number of microcontrollers, a graphics processor unit (GPU), integrated circuits, memory devices (e.g., FLASH, random access memory (RAM), read only memory (ROM), electrically programmable read only memory (EPROM), electrically erasable programmable read only memory (EEPROM), or other suitable variants thereof), and software which co-act with one another to perform operation(s) disclosed herein. In addition, any one or more of the electrical devices may be configured to execute a program code that is embodied in a non-transitory computer readable medium programmed to perform any number of the functions as disclosed.

In the following, embodiments of the present invention will be described in detail with reference to the accompanying drawings. It is to be understood that the following description of embodiments is not to be taken in a limiting sense. The scope of the present invention is not intended to be limited by the embodiments described hereinafter or by the drawings, which are taken to be illustrative only.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Hereinafter, techniques of assisting medical practitioners in drawing up medical reports are presented. Specifically, the techniques disclosed herein include posing one or more diagnostic cues to a user. The one or more diagnostic cues are based on a medical imaging dataset of a current examination of the patient. The one or more diagnostic cues may be optionally based on at least one prior medical report of at least one prior medical examination.

The one or more diagnostic cues can be associated with patient-specific diagnostic findings. For example, the diagnostic cues could be formulated/posed as questions, reports, snippets, etc. The diagnostic cues can be comparably short sentences that convey information content.

As a general rule, the one or more diagnostic cues could point towards - i.e., include a semantic content associated with - or query characteristic features associated with a pathology or abnormalities included in a medical imaging dataset. For instance, diagnostic cues could point towards or query diagnostic root causes of such abnormality. For instance, diagnostic cues could point towards or query follow-up examinations and/or treatments.

The diagnostic cues can be patient specific. I.e., the diagnostic cues can be determined to relate to a patient-specific characteristics. Different examinations with different patients may be associated with different diagnostic cues.

Posing one or more such diagnostic cues can help to engage in an interaction with the user, so as to ensure that the medical report is comprehensive and error-free.

Specifically, the medical report can be generated and/or revised (edited) taking into account the one or more diagnostic cues. This may be based on an automated algorithmic implementation, or based on an appropriate configuration of a user interface (UI) that enables the medical practitioner to take into account the one or more diagnostic cues.

As a general rule: Medical reports could be radiology reports or other text-based electronic medical data such as lab-diagnostics results, clinical notes, surgical records, discharge records, and pathology reports.

To pose the one or diagnostic cues, the UI can be controlled. A graphical UI can be controlled. In particular, the one or more diagnostic cues can be posed as part of the workflow for drawing up the medical report. The workflow for drawing up the medical report can include an interaction framework for the medical practitioner to include relevant information in the medical report. For instance, it would be possible that the one or more cues are linked to certain sections of the medical report that has been pre-generated (if the one or more diagnostic cues are posed to the user prior to a generation step of the workflow). The medical report may be shown face-to-face with the one or more diagnostic cues. The one or more diagnostic cues may be graphically associated with the sections of the medical report.

Thereby, an interactive process can be triggered that enables the medical practitioner to comprehensively and accurately draw up the medical report based on the semantic guidance provided by the one or more diagnostic cues.

As a general rule, there are various options available for implementing said posing of the one or more diagnostic cues. Some options are summarized in TAB. 1 below.

TABLE 1
Various options for implementing diagnostic cues.
The options can also be combined with each other.

| | Example | |
|---|---|---|
| | implementation | Example details |
| I | Question | The diagnostic cues can be posed as |
| | | questions. This means that the user can |
| | | have the possibility to answer a |
| | | medical question via the UI. The method |
| | | may accordingly include obtaining, from |
| | | the UI, a user feedback to at least one |
| | | of the one or more medical cues. Thus, |
| | | additional information relevant for |
| | | drawing up the medical report may be |
| | | collected. This user feedback may be |
| | | used to edit the medical report. |
| | | For instance, “yes/no” questions may be |
| | | posed. It would be possible to acquire |
| | | quantitative feedback, e.g., pertaining |
| | | to the size of certain anatomical |
| | | features. |
| | | It would be possible that user feedback |
| | | is obtained via the UI in response to |
| | | posing questions. This user feedback |
| | | may then be used as part of the |
| | | workflow for drawing up the medical |
| | | report. The medical report may be |
| | | edited based on the user feedback. For |
| | | instance, it would be possible that the |
| | | medical report is generated based on |
| | | the user feedback. Here, the one or |
| | | more questions can be posed to the user |
| | | prior to a generating step of the |
| | | workflow for drawing up the medical |
| | | report. Also, in scenarios where the |
| | | medical report has been pre-generated, |
| | | it would be possible that the medical |
| | | report is revised based on the user |
| | | feedback. |
| II | Notes | It would be possible that the |
| | | diagnostic cues are posed as notes. |
| | | Here, it is not required to obtain |
| | | explicit user feedback. Rather, the |
| | | user may implicitly consider the notes |
| | | and use them, as deemed appropriate. |
| | | For instance, the notes can be used as |
| | | checklist or “safety net”. |
| | | The diagnostic cues can, accordingly, |
| | | provide context information that can be |
| | | helpful to the medical practitioner |
| | | when drawing up the medical report. For |
| | | instance, it would be possible that the |
| | | one more diagnostic cues are posed as |
| | | notes after a generation step over the |
| | | workflow for drawing up the medical |
| | | report. Thereby, a validation of the |
| | | medical report—e.g., for completeness |
| | | and/or correctness—may be facilitated |
| | | by the one or more diagnostic cues. |
| | | The one or more diagnostic cues could |
| | | be determined based on the medical |
| | | report that has been pre-generated. |
| | | I.e., it would be possible that the one |
| | | or more diagnostic user tailored to the |
| | | medical report. Thereby, for instance, |
| | | when the user manually draws up the |
| | | medical report, during the validation |
| | | step, the user can be confronted with |
| | | the one or more diagnostic cues that |
| | | act as a checklist for completeness or |
| | | serve to cross checked the correctness |
| | | of content of the medical report. |
| | | Based on the one or diagnostic cues, |
| | | the medical practitioner may check |
| | | whether all information is contained in |
| | | the medical report, or with additional |
| | | information is required. The medical |
| | | practitioner may check whether the one |
| | | or diagnostic cues are consistent or |
| | | inconsistent with the medical report. |
| | | Thereby, the validation step of the |
| | | workflow may provide for a validation |
| | | toolset for the medical practitioner to |
| | | validate the medical report. |

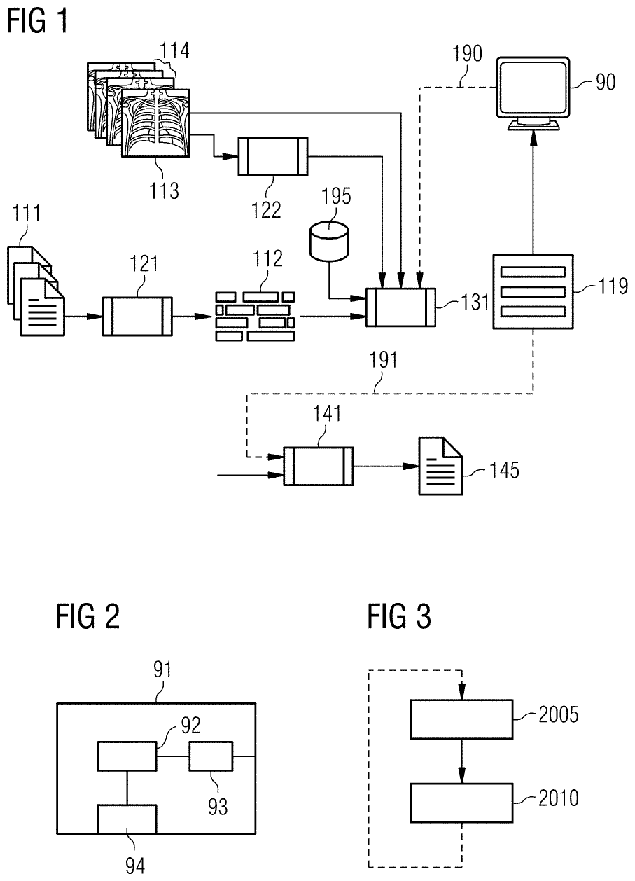

FIG. 1 schematically illustrates data processing according to various examples. FIG. 1 illustrates aspects with respect to one or more cues 119 that are posed to a user as part of a workflow for drawing up a current medical report 119. The one or more cues 119 are determined using a processing algorithm

As illustrated in FIG. 1, at least one prior medical report 111 of at least one prior examination of the patient is obtained (this is, however, generally optional). For instance, the at least one medical report 111 could be retrieved from a medical repository of the hospital. It could be retrieved from a server.

Using the at least one prior medical report 111 of the at least one prior examination in connection with drawing up the current medical report facilitates a longitudinal analysis. For instance, the development of a pathology of the patient may be assessed. In particular, the one or more diagnostic cues can pertain to such development of a pathology between the at least one prior examination and the current examination.

Then, using a natural language processing algorithm 121, the at least one medical report 111 may analyzed to obtain a machine-readable representation 112 of the at least one prior medical report. This serves as an input to the processing algorithm 131. As a general rule, using the natural language processing algorithm 121 is optional. It would be possible that the processing algorithm 131 directly operates based on the at least one prior medical report 111.

The processing algorithm 131 also obtains, as an input, a current medical imaging dataset 113 or result data that is determined by a medical analytics algorithm 122 based on the current medical imaging dataset 113. The processing algorithm 131 thus also operates based on a medical imaging dataset 113 of the current examination of the patient. Alternatively or additionally to the current medical imaging dataset 113, it would also be possible to obtain at least one medical imaging dataset 114 of the at least one prior examination (e.g., the same at least one prior examination to which the at least one prior medical report 111 pertains to, or another prior examination). The one or more diagnostic cues 119 can be determined further based on the at least one prior medical imaging dataset.

Illustrated in FIG. 1 are two options for taking into account the current medical imaging dataset 113 and/or the at least one prior medical imaging dataset 114. In one option, it is possible that the processing algorithm 131 that determines the one or more diagnostic cues 119 directly operates based on the respective imaging dataset or datasets 113, 114. Alternatively or additionally, it would also be possible to analyze the current medical imaging dataset 113 and/or the at least one prior medical imaging dataset 114 using a medical analytics algorithm 122 to determine result data for the respective medical imaging dataset. Then, the one or more diagnostic cues 119 can be determined based on the result data.

The current and/or prior medical imaging dataset could be one or more of the following: magnetic resonance tomography images; computed tomography images; OCT images; images obtained by photoacoustic tomography; to determine emission tomography images; SPECT images; images obtained from diffuse scattering tomography; infrared tomography images; ultrawide bandwidth tomography images; ultrasound images; echocardiogram data; images of histopathology samples; stained tissue samples; etc. The techniques can apply to radiology or other disciples such as oncology, dermatology, ophthalmology etc. and is not limited to radiological images.

The processing algorithm 131 can, optionally, take into account one or more parameters. Such parameters can be obtained from a database 195. One example from each of the be an institution-specific reporting guidelines for drawing up medical reports. In other words, it would be possible that the processing algorithm 131 determines the one or diagnostic cues 119 in accordance with the institution-specific reporting guidelines for drawing up medical reports. Respective parameterization data can be obtained from the database 195 and fed to a respective input channel of the processing algorithm 131. It would also be possible that the processing algorithm 131 is fixedly trained to determine the one or more diagnostic cues 119 in accordance with a single institution-specific reporting guideline. Thereby, the processing algorithm 119 can also be built on Clinical Reporting Guidelines for individual modalities such as Bi-RADS, Pi-RADS, LungRADS to name a few.

Once the processing algorithm 131 has determined the one or more diagnostic cues 119, it is then possible to control a UI 90 as part of a workflow for drawing up the current medical report 145. The UI 90 is controlled to pose the one or more diagnostic cues (cf. TAB. 1).

For instance, the posing can be triggered in the backend as soon as a particular case is sent to the radiology worklist. Once the case is opened for reporting, the one or more diagnostic cues 119 can either be shown on the PACS/RIS window, or as a pop-up message or before case-closeout as a confirmatory step. Alternatively, posing the one or more diagnostic cues can be triggered on-demand in response to one or more trigger criteria. For instance, posing the one or more cues can be triggered if it is judged that diagnostic errors/blanks exist in the medical report 145; then an associated diagnostic cue can be posed to the user. Where multiple diagnostic cues 119 can be posed to the user, it would be possible that the multiple diagnostic cues 119 art posed all in parallel to the user. It would also be possible that the multiple cues are sequentially posed to the user.

In some examples, it would be possible that the multiple cues 119 are sequentially determined. Specifically, it would be possible to obtain a user feedback associated with a first one of the multiple cues. Then, a second one of the multiple cues can be determined, using the processing algorithm 131 considering the user feedback (cf. FIG. 1, user feedback 190).

Depending on the implementation, it would then be possible that the user interactively considers these one or more diagnostic cues 190 when drawing up the medical report 145. It would also be possible that user feedback 191 is obtained, e.g., where the one or more diagnostic cues are implemented as questions. Then, using an appropriate algorithm 141, the medical report 145 may be generated or, at least, revised based on the user feedback 191. Here, further input may be considered, e.g., the medical imaging dataset 113 and/or at least one prior medical report 111.

Summarizing, via the data processing of FIG. 1, a radiology question generation engine can be implemented which takes in unstructured free text of past radiology reports 111, prior images 114 and a current image 113 and provides a list of actionable questions 119 to be posed to the radiologist while reporting on the current image. Thereby, a more accurate and comprehensive diagnosis can be facilitated. The one or more diagnostic cues 119 also support medical education by acting as a virtual pedagogical teacher posing pertinent questions to junior radiologists/residents. It can aid in clinical decision making and in patient education to help patients pose the right clinical questions to their clinicians.

FIG. 2 schematically illustrates a device 91 that can implement the processing according to FIG. 1, at least in parts. The device 91 includes a processor 92 in the memory 93, as well as an interface 94. For instance, the processor 92 can receive medical imaging datasets 113, 114 and/or at least one prior medical report 111 via the interface 94. The processor 92 can load and execute program code from the memory 93. Loading and executing the program code causes the processor 92 to perform techniques as disclosed herein, e.g., techniques associated with executing a workflow for drawing up medical report, controlling a UI 90, posing one more diagnostic cues 119 that have been determined based on executing a processing algorithm 131, image preprocessing using a medical analytics algorithm 122, editing a medical report, training a processing algorithm for determining one or more diagnostic cues, etc.

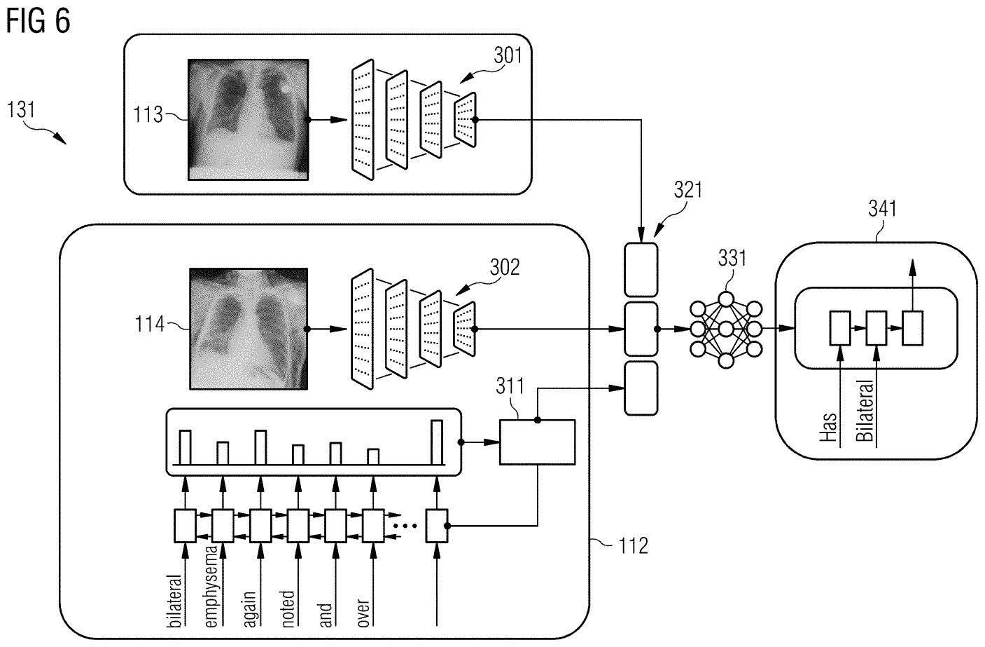

As a general rule, various options are available for implementing the processing algorithm 131. Specifically, it would be possible that the processing algorithm 131 is implemented as a machine-learning algorithm. Here, in a training phase 2005 (cf. FIG. 3; further details will be explained in connection with FIG. 6 below for a specific example implementation of the processing algorithm 131), the machine-learning processing algorithm 131 can be trained. For this, appropriate training data can be considered, the training data including a training input and a ground-truth label. Then, using techniques such as gradient descent optimization and/or back propagation, it is possible to adjust weights of the machine-learning processing algorithm 131, during the training phase 2005.

Once the machine-learning processing algorithm 131 has been appropriately trained, it is possible to implement an inference phase 2010. Here, the one or diagnostic cues 119 can be predicted based on input data as discussed above in connection with FIG. 1.

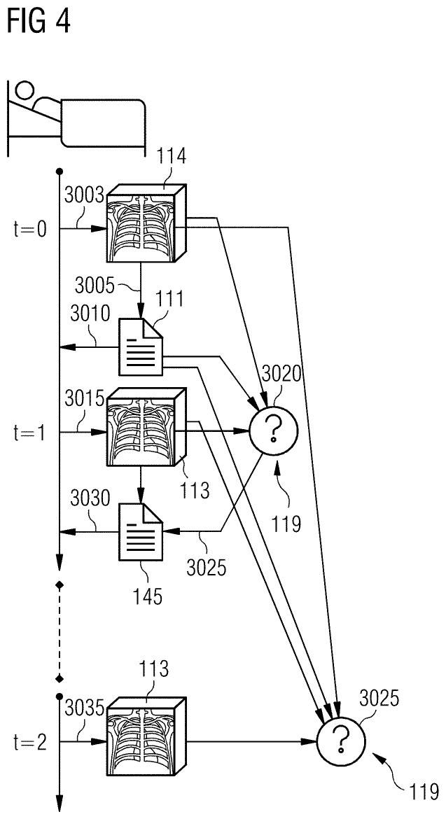

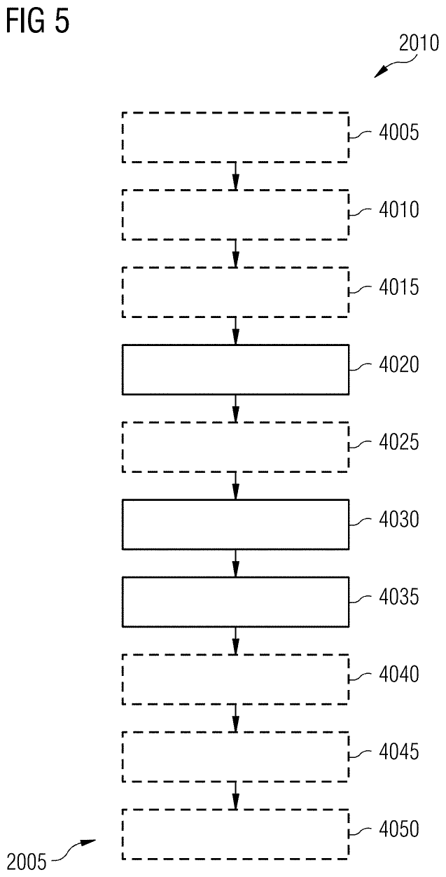

In some examples, the training phase 2005 may be re-executed from time to time, based on ground-truth labels obtained from or during the inference phase 2010. This is a technique that can be labeled life-long-learning (LLL). It is illustrated in FIG. 3 using the dashed feedback arrow. FIG. 4 schematically illustrates a diagnostic workflow according to various examples. Various actions are resolved over time (from top to bottom in FIG. 4).

At 3003, a medical imaging dataset 114 is acquired (at time t=0).

According to the examples disclosed herein, a medical imaging dataset could include one or more of the following: an x-ray image; an ultrasound image; a blood analysis; a urine analysis; a computed tomography image; a magnetic resonance tomography image; to give just a few examples.

At 3005, a medical report 111 is drawn up based on the medical imaging dataset 114, e.g., manually by a medical practitioner or aided by an algorithm (cf. FIG. 1, algorithm 141). For instance, the medical report 111 can be drawn up by a first radiologist.

The medical report 111 is stored at 3010.

At a later point in time (t=1), at 3015, the current medical imaging dataset 113 is acquired.

Based on this, at 3020, one on more diagnostic cues that are associated with patient-specific diagnostic findings determined based on the current medical imaging dataset 113 are posed to the user, e.g., a second radiologist. This occurs during a workflow for drawing up, at 3025, a current medical report 145.

The current medical report 145 is then stored at 3030.

The process can be again repeated at 3025 using a further current medical imaging dataset 113.

FIG. 5 is a flowchart of a method according to various examples. The method of FIG. 5 facilitates support of a medical practitioner when drawing up a medical report. The method of FIG. 5 can implement, e.g., the data processing according to FIG. 1. The method of FIG. 5 can implement parts of the workflow of FIG. 4, e.g., those parts for drawing up the medical report 145 at 3015, 3020, 3025, 3030.

Optional boxes are labeled with dashed lines in FIG. 5.

For instance, the method of FIG. 5 could be executed by a processing device such as the device 91. For instance, the processing of FIG. 5 could be implemented by the processor 92 upon loading program code from the memory 93 and upon executing the program code.

At optional box 4005, at least one prior medical report of at least one prior examination of a patient is obtained. It would be possible to obtain a series of prior medical reports of the patient, e.g., as discussed in connection with FIG. 4. A longitudinal analysis can thereby be facilitated.

It would be possible to obtain the at least one prior medical report from an Electronic Health Records, from a hospital information system, from a radiology information system, to give just a few examples.

Medical reports can be written free text and are typically organized into multiple sections such as background, findings, and impression.

The at least one prior medical report may be loaded via an interface, e.g., the interface 94 of the device 91 (cf. FIG. 2).

At optional box 4010, it is then possible to use a natural language processing algorithm to analyze the at least one prior medical report to obtain a machine-readable representation of the at least one prior medical report (cf. FIG. 1): machine-readable representation 112. For instance, it would be possible that the machine-readable representation classifies multiple sections of the at least one prior medical report. This, later on, facilitates providing different ones of the multiple sections to different input channels of the processing algorithm that is used to determine the one or more diagnostic cues.

As a general rule, various options are available for preprocessing, using the natural language processing algorithm, the at least one prior medical report. For instance, the at least one prior medical report may be tokenized and parsed into sections corresponding to findings, backgrounds, indications, and summary.

This can be achieved using techniques such as named entity recognition, parts-of-speech tagging, lower-casing and removing stop words. Named entities can be optionally replaced with respective tags (such as RadLex mapping/CT-SNOMED mapping) to allows for better model generalization and learn patterns in the data.

An example is shown in TAB. 2.

TABLE 2
Example parsing and tokenization of
free text in a prior medical report.

| | |
|---|---|
| “Bilateral emphysematous | “[[RID5771]] [[RID4799]] |
| again noted and lower lobe | again noted and [[RID34696]] |
| fibrotic changes. | [[RID3820]] changes. |
| Postsurgical changes of the | Postsurgical changes of the |
| chest including cabg | [[RID1243]] including |
| procedure, stable.” | [[RID35862]] procedure, |
| | stable.” |

To enable knowledge transfer from a large corpus of related medical data, at box 4010 it is possible to use word vectors from methods such as GloVE (Pennington, J., Socher, R. and Manning, C. D., 2014, October. Glove: Global vectors for word representation. In Proceedings of the 2014 conference on empirical methods in natural language processing (EMNLP)

(pp. 1532-1543)), or language-models such as Bio-BERT (Lee, J., Yoon, W., Kim, S., Kim, D., Kim, S., So, C. H. and Kang, J., 2020. BioBERT: a pre-trained biomedical language representation model for biomedical text mining. Bioinformatics, 36(4), pp. 1234-1240.) to name a few.

At optional box 4015, it is possible to obtain at least one prior medical imaging dataset (cf. FIG. 1: prior medical imaging datasets 114).

It would be possible to load such at least one prior medical imaging dataset from a picture archiving system or, generally, from a patient data repository.

At box 4020, it is then possible to obtain a current medical imaging dataset that is associated with a current examination of the patient. The current medical imaging dataset serves as the basis for the medical report to be drawn up.

It would be possible to load such at least one current medical imaging dataset from a picture archiving system or, generally, from a patient data repository. A medical imaging device may be controlled to acquire the current medical imaging dataset.

At optional box 4025, it would be possible to preprocess/analyze all or at least one of the available medical imaging datasets, using an appropriate medical analytics algorithm. Respective techniques have been discussed in connection with FIG. 1: medical analytics algorithm 122.

Various medical analytics algorithms are known. Such medical analytics algorithms can extract one more quantitative or qualitative properties from medical imaging datasets. For instance, medical analytics algorithms are known that detect liaisons or tumors. Medical analytics algorithms are known that detect abnormal anatomic conditions. The particular implementation of the medical analytics algorithm is not germane for the functioning of the techniques disclosed herein; as such, prior art implementations of medical analytics algorithms can be used.

Then, at box 4030, it is possible to determine one or more diagnostic cues that are associated with patient-specific diagnostics findings. This is based, at least, on the medical imaging dataset that is obtained at box 4020, and potentially the output of the medical analytics algorithm executed at box 4025. Optionally, the determining of the one or more diagnostic cues could also be based on the at least one prior medical report as obtained in box 4005 and as optionally preprocessed in box 4010.

In some examples, the current medical report can be pre-generated, e.g., manually by the user. In such examples, it would be possible that the one or more diagnostic cues are determined based on the pre-generated current medical report. Note that even though the current medical report has been pre-generated, it is possible to subsequently revise the pre-generated current medical report, based on the one or more diagnostic cues.

At box 4035, the one or more diagnostic cues are posed to the user. A UI can be controlled accordingly.

Optionally, at box 4040 user feedback regarding the semantic content of the one or more diagnostic cues can be obtained, e.g., the one or more cues can be posed as questions

(cf. FIG. 1: feedback 190, 191).

It would be optionally possible that the medical report is generated and/or revised (edited) based on user feedback of box 4040. In other examples, it would also be possible that the user manually draws of the medical report. Optionally, at box 4050, LLL could be implemented, as part of the training phase 2005. This can be based on further user feedback regarding a quality of the one or more cues that are posed to the user at box 4035. A “like” or “dislike” button may be used. A “1 to 5 star rating” or the like may be used. Thereby, ground-truth labels for the training can be derived. Various options are available for implementing the processing algorithm used at box 4030 to determine the one or more diagnostic cues. An example implementation as illustrated in FIG. 6.

FIG. 6 schematically illustrates an example implementation of the processing algorithm 131 used to determine one or more diagnostic cues. In the example of FIG. 6, the processing algorithm 131 is a machine-learning algorithm. This means that weights/parameter values of the machine-learning algorithm can be set in a training process 2005 using optimization techniques based on a ground-truth label.

The machine-learning algorithm 131 is based on a neural-network architecture. The processing algorithm 131 includes multiple encoder branches 301, 302, 311. Each encoder branch determines respective latent features 321.

The encoder branches can each include multiple subsequent layers for contracting the dimensions of the input data (encoding).

For instance, the encoder branch 301 determines latent features 321 for the current medical imaging dataset 113.

For instance, the encoder branch 302 determines latent features 321 for the previous medical imaging dataset 114.

For instance, the encoder branch 311 determines latent features 321 for the at least one prior medical report. In the illustrated example, the encoder branch 300 operates based on a machine-readable representation 112. For instance, a recurrent neural network (RNN) such as a bi-directional long-short-term memory encoder branch 311 may be used for determining the latent features 321 of the machine-readable representation 112.

Within the RNN-encoder, an attention layer can be used (not illustrated in FIG. 6). The attention layer can determine shortcuts between respective latent features 321 and a respective input to that encoder branch. Such attention mechanism may be implemented in accordance with: Yu, Z., Yu, J., Cui, Y., Tao, D. and Tian, Q., 2019. Deep modular co-attention networks for visual question answering. In Proceedings of the IEEE/CVF Conference on Computer Vision and Pattern Recognition (pp. 6281-6290).

Thereby, the diagnostic cue generation can focus on appropriate named entities and relationships; rather than on sematic variations due to individual reporting styles.

The encoder branches 301, 302 can be implemented as a convolutional neural network (CNN). Here, in convolutional layers, input weights of convolved with a predefined kernel for each layer. The weights of the kernel are trained in the training phase 2005.

The CNN encoder can be represented using deep CNNs such as deep belief network, ResNet, DenseNet, Autoencoders, capsule networks, generative adversarial networks, Siamese networks, convolutional neural networks, variational autoencoders, image transformer networks etc.

To ensure that the generated cues are intelligible, templates can be used. Some question templates are shown in TAB. 3 below.

TABLE 3
example cues templates, here for an implementation of posing
the cues as questions as in TABLE 1 example I. #### is
the placeholder for tokenized named entities. The entities
in ### may be either filled using copying mechanisms-
see, e.g., Gu, J., Lu, Z., Li, H. and Li, V.O., 2016. Incorporating
copying mechanism in sequence-to-sequence learning. arXiv preprint
arXiv:1603.06393. - or use pointer generation networks-see,
e.g., See, A., Liu, P.J. and Manning, C.D., 2017. Get to the
point: Summarization with pointer-generator networks. arXiv
preprint arXiv:1704.04368.

| | Example question template |
|---|---|
| I | What imaging abnormalities can be seen in ###? |
| II | What is the most likely cause of ### abnormality? |
| III | What is the most appropriate follow-up imaging |
| | modality for monitoring ###? |
| IV | Is there a ### in the ###? |

Generally, instead of posing the cues as questions, the cues may be posed as notes. This is shown in TAB. 4 for the similar examples as in TAB. 3.

TABLE 4
example cues templates, here for an implementation of
posing the cues as questions as in TABLE 1 example II.

| | Example note template |
|---|---|
| I | The imaging abnormality ### can be seen. |
| II | The most likely root cause for ### is XYZ. |
| III | XYZ is the most likely follow-up imaging modality for |
| | monitoring ###. |
| IV | There is a ### in the ###? |

FIG. 6 illustrates a scenario in which the preprocessing algorithm 131 obtains the medical imaging datasets 113, 114 as inputs. As explained in connection with FIG. 1: it would be possible, to use a medical analytics algorithm 122 to preprocess the images and then provide the output of the medical analytics algorithm 122 as a further input to the processing algorithm 131 (or provide the output of the medical analytics algorithm 122 instead of the medical imaging datasets 113, 114 is input).

Thus, the processing algorithm 131 can optionally take in automated results generated using medical analytics algorithm. For example, given a chest X-ray image, the medical analytics algorithm can detect and characterize radiographic findings within the image. If there are any new emergent suspicious radiographic findings that were not seen in the previous timepoint image or report, this can be flagged as a diagnostic cue to the reporting radiologist.

Once the latent features 321 have been determined, the latent features can be fused in a fusing layer 331. Then, a decoder branch 341 can be used to determine the one or diagnostic cues. Thus, the latent features of multiple encoder branches can be merged to obtain merge latent features and then, a decoder branch can be used for reconstructing the one or more diagnostic cues based on the merged latent features.

As will be appreciated, where the processing algorithm 131 considers one or prior medical reports and outputs the one or diagnostic cues, the processing algorithm can be implemented by a sequence-to-sequence network, combined with an image-to-sequence network.

As a general rule, it would be possible that a fixed number of diagnostic cues is output by the processing algorithm. A random variable provided as a control input to the processing algorithm can introduce variability between different diagnostic cues. An index can toggle between different diagnostic cues. Generally, user feedback obtained in response to opposing a first diagnostic cue to the user can be used as an input for determining a subsequent, second diagnostic cue (cf. FIG. 1: feedback 190).

The processing algorithm 131 thus can be described as follows:

Given a tokenized prior medical report X_{T-1}=(x₁. . . xₙ) (cf. TAB. 2), a prior medical imaging dataset including, e.g., an image I_{T-1} and a current medical imaging dataset including, e.g., a current image I_{T}, the processing algorithm 131 generates cues Y_{T}=(y₁. . . yₘ) which ae defined as the best cues Ŷ_{T} that maximizes the condition likelihood given X_{T-1}, I_{T-1}, I_{T}:

$${\hat{Y}}_{T} = arg {max}_{Y} P ( Y \vert {X}_{T - 1} , {I}_{T - 1} , {I}_{T} ) = arg {max}_{Y} {\sum }_{t = 1}^{m} P ( {y}_{t} \vert {X}_{T - 1} , {I}_{T - 1} , {I}_{T} , {y}_{< t} )$$

P can be modeled using a hybrid architecture with an RNN-based encoder for modeling X_{T-1}, CNN-based encoder for I_{T-1} and I_{T} and an RNN-based decoder for Ŷ_{T}.

The training phase 2005 (cf. FIG. 3) of such processing algorithm can be implemented as follows:

Multiple training datasets can be obtained. Each training dataset can include respective training medical imaging datasets and optionally respective training medical reports.

Training datasets can be obtained for different imaging modalities and/or different imaged anatomical regions. Training datasets can be obtained for different imaging equipment. The training datasets can be harmonized to minimize inter-site variations. Quality control can be implemented based with expert medical professionals. The training datasets could be anonymized. The training medical imaging datasets can be matched to the training medical reports. This corresponds to various inputs to the processing algorithm 131, as discussed in connection with FIG. 6.

Then, experts can manually determine diagnostic cues for the multiple training datasets. This corresponds to ground-truth label generation. For instance, actionable questions can be selected by multiple experts from relevant question templates to generate a corpus of expert verified report—question pairs.

By this, pairs of input and output data as ground truth for the processing algorithm 131 are obtained, for the training datasets.

Then, the training can be implemented using conventional training techniques, e.g., gradient descent and backpropagation. Loss functions such as cross-entropy loss, KL-divergence, question reconstruction loss and regularization loss functions can be used.

The trained processing algorithm could then be validated, e.g., based on unseen-held-out training datasets. Various validation metrics are conceivable, e.g., BLEU, METEOR, ROGUE etc.

In some examples, LLL can be employed (cf. FIG. 3, feedback loop; FIG. 5: box 4050). This means that posing the one or more diagnostic cues to the user during the inference phase 2010 can be operated in a closed loop with the training phase 2005 (cf. FIG. 3). The ground-truth labels could be either explicit radiologist feedback such a satisfaction with generated questions, ‘like’ button or scoring like a satisfaction scale. Alternatively, or additionally, indirect performance and quality metrics would be concise such as reduced hedging, detailed reports, improved quality of reporting in anonymous peer review etc. Such a feedback can be used to improve the question generation using methods for weak supervision, reinforcement learning etc. thus, it would be possible to obtain user feedback associated with the one or more diagnostic cues. The user feedback can indicate a quality or relevance of each one of the one or diagnostic cues. Then, at least a part of the processing algorithm 131 can be retrained based on a user feedback.

Next, further details with respect to taking into account one or more prior medical reports will be discussed in connection with the following FIGS.

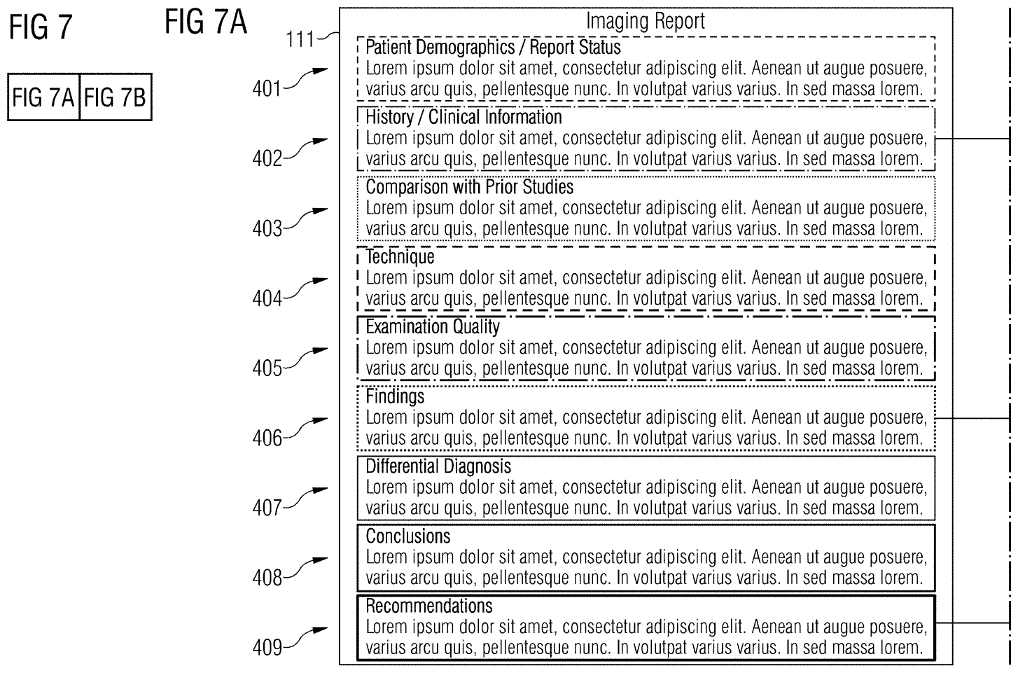

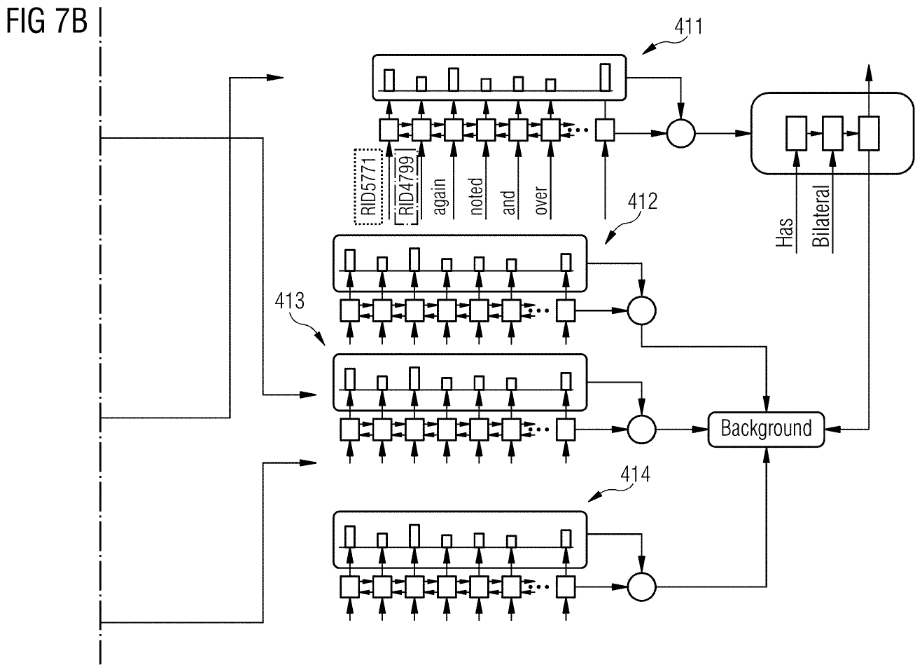

FIG. 7 illustrates aspects with respect to the prior medical report 111. The prior medical report includes multiple sections such as a patient demographics section 401, a clinical history section 402 of the patient, a section 403 pertaining to a comparison of the medical examination subject to the prior medical report 111 with one or more further prior examinations, a technique section 404, a section 405 outlining the quality of the medical examination underlying the prior medical report 111, a findings section 406, a diagnosis section 407, a conclusion section 408, and a recommendation section 409.

Other medical reports may include other sections, or fewer sections, or additional sections. FIG. 7 is only an example.

FIG. 7 also illustrates aspects with respect to processing the multiple sections 401-409 using the processing algorithm 131.

As a general rule: Multiple encoder branches 411-414 can be used to process different sections of the prior medical report 111, e.g., a machine-readable representation thereof. The different encoder branches may be trained separately or jointly.

The different sections can thus, generally, provided to different input channels of the processing algorithm 131.

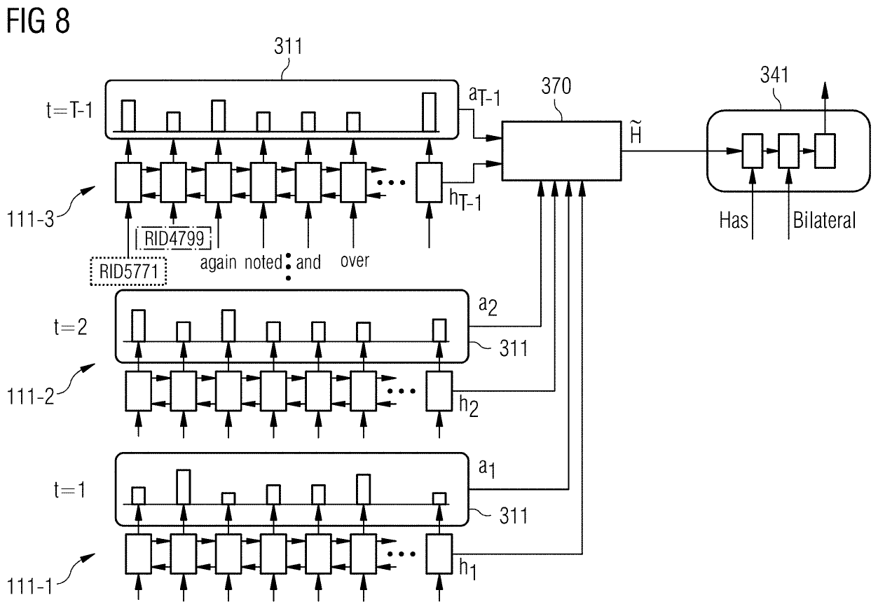

Above, in connection with FIG. 6 and FIG. 7, scenarios have been explained in which a single prior medical report 111 (or, respectively, the machine-readable representation 112 thereof) is used as an input to the processing algorithm 131. As a general rule (and as explained at high generality in connection with FIG. 4), it would be possible to obtain multiple prior medical reports of multiple prior examinations of the patient. It would be possible that the processing algorithm 131 processes all these multiple prior medical reports. A corresponding example is illustrated in FIG. 8.

FIG. 8 illustrates aspects with respect to the processing algorithm 131 processing multiple prior medical reports 111-1, 111-2, 111-3. Here, three prior reports 111-1-111-3 are obtained, for multiple prior time points. Each prior medical report is processed in a respective instance of the encoder branch 311. Then, a multiple-instance pooling layer is 370 used to merge the respective latent features encoding each one of the prior reports 111-1-111-3 using the encoder branch 311.

The multiple-instance pooling facilitates fusion of latent features associated with an arbitrary number of prior medical reports.

For instance, in typical routine chest X-ray monitoring scenarios, a series of medical reports and images are collected over time. The knowledge from all the prior scans (t<T) can be used as input to the processing algorithm 131 in timepoint T.

This can be implemented using multiple-instance learning. The multiple-instance pooling layer 370 can be realized using differentiable pooling functions such as Noisy-OR, log-sum exponentiation, max-pooling, softmax pooling, noisy-AND pooling, generalized mean-pooling, etc.

The medical-instance pooling layer can include attention weighting for the multiple medical reports 111-1-111-3.

Hence, it is possible to combine an attention mechanism with multiple instance pooling. The hidden state of the report encoders for predict at time T can be represented as H=h₁. . . . h_{T-1}) and the corresponding attention matrix as A=(a₁. . . . a_{T-1}). The hidden vectors fed into the RNN-decoder 341 for question generation are defined as \tilde{H}=max_{T}A⊙H where ⊙ is element-wise multiplication and max operator. Alternative mechanisms such as time-base attention, interaction base attention etc. can also be used, see, e.g., Ma, F., Chitta, R., Zhou, J., You, Q., Sun, T. and Gao, J., 2017, August. Dipole: Diagnosis prediction in healthcare via attention-based bidirectional recurrent neural networks. In Proceedings of the 23rd ACM SIGKDD international conference on knowledge discovery and data mining (pp. 1903-1911).

Summarizing, various techniques have been disclosed which facilitate drawing up a medical report by a medical practitioner. This is based on one more diagnostic cues that are posed to the user during a workflow for drawing up the medical report.

Optionally, such one or more diagnostic cues may also be posed to a patient so that the patient can use the one or more diagnostic cues to analyze what the right questions is to ask their caregiver.

Optionally, the same technique can be applied to other tasks beyond medical report preparation, e.g., tumor-boards, therapy planning, patient monitoring, screening etc.

Summarizing, at least the following EXAMPLES have been disclosed:

### EXAMPLE 1

### A Computer-Implemented Method, Comprising

obtaining (4020) medical imaging dataset (113) of a current examination of a patient,
based on the medical imaging dataset (113), determining one or more di-agnostic cues (119) using a processing algorithm, the one or more diagnostic cues (119) being associated with patient-specific diagnostic findings, and
as part of a workflow for drawing up a current medical report (145) for the current examination, controlling (4035) a user interface (90) to pose the one or more diagnostic cues (119) to a user.

### EXAMPLE 2

### The Computer-Implemented Method of Example 1

wherein the one or more diagnostic cues (119) are posed as questions,

wherein the method further comprises:

obtaining, from the user interface (90), a user feedback (119) to at least one of the one or more diagnostic cues, and

editing the current medical report (145) based on the user feedback.

### EXAMPLE 3

### The Computer-Implemented Method of Example 2

wherein said editing of the current medical report based on the user feedback comprises generating the medical report or refining the current medical report.

### EXAMPLE 4

### The Computer-Implemented Method of Any One of the Preceding Examples

wherein the user interface (90) is controlled to pose the one or more diagnostic cues (119) to the user prior to a generation step of the workflow.

### EXAMPLE 5

### The Computer-Implemented Method of Any One of Examples 1 to 3

wherein the user interface is controlled to pose the one or more diagnostic cues (119) to the user after a generation step of the workflow as part of which the current medical report is generated and during a validation step of the workflow providing a validation toolset to the user for refining the current medical report (145), wherein the one or more diagnostic cues are optionally determined further based on the current medical report.

### EXAMPLE 6

### The Computer-Implemented Method of Any One of the Preceding Examples, Further Comprising

obtaining (4005) at least one prior medical report (111) of at least one prior examination of the patient, and

using a natural language processing algorithm (121), analyzing the at least one prior medical report (111) to obtain a machine-readable representation (112) of the at least one prior medical report (111),

wherein the one or more diagnostic cues (119) are determined based on the machine-readable representation (112) of the at least one prior medical report (111-3).

### EXAMPLE 7

### The Computer-Implemented Method of Example 6

wherein the machine-readable representation (112-2) of the at least one prior medical report (111) classifies multiple sections (401-409) of the at least one prior medical report (111),

wherein different ones of the multiple sections (401-409) are provided to different input channels of the processing algorithm (131), the different input channels being optionally associated with different encoder branches (411-414) of the processing algorithm (131).

### EXAMPLE 8

### The Computer-Implemented Method of Any One of the Preceding Examples, Further Comprising

analyzing (4025) the medical imaging dataset (113) of the current examination using a medical analytics algorithm (122) to determine result data for the medical imaging dataset (113),

wherein the one or more diagnostic cues (119) are determined based on the result data.

### EXAMPLE 9

### The Computer-Implemented Method of Any One of the Preceding Examples, Further Comprising

obtaining (4015) at least one prior medical imaging dataset (114) of at least one prior examination,

wherein the one or more diagnostic cues (119) are determined further based on the at least one prior medical imaging dataset (114) of the at least one prior examination.

### EXAMPLE 10

### The Computer-Implemented Method of Any One of the Preceding Examples, Further Comprising

obtaining at least one prior medical report (111) of at least one prior examination of the patient,

wherein the processing algorithm is machine-learned and comprises multiple encoder branches (301, 302, 311) configured to determine respective latent features, different encoder branches (301, 302, 311) being associated with the at least one prior medical report and the medical imaging dataset (113) of the current examination.

### EXAMPLE 11

### The Computer-Implemented Method of Example 10

wherein a first encoder branch (311) of the multiple encoder branches associated with the at least one prior medical report (111) comprises a re-current neural network such as a bi-directional long-short-term memory encoder branch.

### EXAMPLE 12

### The Computer-Implemented Method of Example 10 or 11

wherein a second encoder branch (301, 302) of the multiple encoder branches associated with the medical imaging dataset (113) of the current examination comprises a convolutional neural network.

### EXAMPLE 13

### The Computer-Implemented Method of Any One of Examples 10 to 12

wherein at least one encoder branch (311-3) of the multiple encoder branches associated with the at least one prior medical report comprises an attention layer for determining shortcuts between the respective latent features (321) and a respective input to the processing algorithm (131).

### EXAMPLE 14

### The Computer-Implemented Method of Any One of Examples 10 to 13

wherein the processing algorithm (131) merges (331) the latent features (321) of the multiple encoder branches (301, 302, 311), to obtain merged latent features,

wherein the processing algorithm (131-4) comprises a decoder branch (341) for reconstructing the one or more diagnostic cues based on the merged latent features.

### EXAMPLE 15

### The Computer-Implemented Method of Any One of Examples 10 to 14, Further Comprising

obtaining multiple prior medical reports (111) of multiple prior examinations of the patient and wherein the processing algorithm (131) comprises a multiple-instance pooling layer (370) to merge latent features obtained from a respective encoder branch (311) of the multiple encoder branches used to encode each one of the multiple prior medical reports (111).

### EXAMPLE 16

### The Computer-Implemented Method of Example 15

wherein the multiple-instance pooling layer (370) comprises attention weighting for the multiple prior medical reports (111).

### EXAMPLE 17

### The Computer-Implemented Method of Any One of Examples 10 to 15, Further Comprising

obtaining (4050) a user feedback associated with the one or more diagnostic cues, and

re-training at least a part of the processing algorithm (131) based on the user feedback.

### EXAMPLE 18

### The Computer-Implemented Method of Any One of the Preceding Examples

wherein multiple diagnostic cues (1190) are sequentially determined,

wherein the method further comprises: obtaining a user feedback (190) associated with the multiple cues,

wherein said controlling of the user interface (90), said determining of the multiple diagnostic cues, and said obtaining of the user feedback (190) is implemented in an entangled manner, so that a subsequent diagnostic cue (119) of the multiple diagnostic cues (119) depends on the user feedback (190) associated with a preceding cue (119).

### EXAMPLE 19

### The Computer-Implemented Method of Any One of the Preceding Examples

wherein the processing algorithm (131-) determines the one or more diagnostic cues (119) in accordance with an institution-specific reporting guideline for drawing up medical reports.

### EXAMPLE 20

A device (91) comprising a processer (92) configured to perform the method of any one of the preceding Examples.

### EXAMPLE 21

### A Computer Program or a Computer Program Code that is Executable by a Processor (92), wherein Execution of the Program Code Causes the Processor to

obtain (4020) medical imaging dataset (113) of a current examination of a patient,

based on the medical imaging dataset (113), determine one or more diagnostic cues (119) using a processing algorithm, the one or more diagnostic cues (119) being associated with patient-specific diagnostic findings, and

as part of a workflow for drawing up a current medical report (145) for the current examination, control (4035) a user interface (90) to pose the one or more diagnostic cues (119) to a user.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain preferred embodiments, equivalents and modifications will occur to others skilled in the art upon the reading and understanding of the specification. The present invention includes all such equivalents and modifications and is limited only by the scope of the appended claims.

What is claimed is:

1. A computer-implemented method, comprising:
obtaining a medical imaging dataset of a current examination of a patient;
determining, based on the medical imaging dataset, one or more diagnostic cues using a processing algorithm, the one or more diagnostic cues being associated with patient-specific diagnostic findings; and
controlling a user interface to pose the one or more diagnostic cues to a user, as part of a workflow for drawing up a current medical report for the current examination.

2. The computer-implemented method of claim 1,
wherein the one or more diagnostic cues are posed as questions, and
wherein the computer-implemented method further includes
obtaining, from the user interface, user feedback to at least one of the one or more diagnostic cues, and
editing the current medical report based on the user feedback.

3. The computer-implemented method of claim 2, wherein said editing of the current medical report based on the user feedback comprises:
generating the current medical report or refining the current medical report.

4. The computer-implemented method of claim 1, wherein the controlling controls the user interface to pose the one or more diagnostic cues to the user after generating the current medical report and during validation of the workflow providing a validation toolset to the user for refining the current medical report.

5. The computer-implemented method of claim 1, further comprising:
obtaining at least one prior medical report of at least one prior examination of the patient; and
analyzing, using a natural language processing algorithm, the at least one prior medical report to obtain a machine-readable representation of the at least one prior medical report, wherein
the determining determines the one or more diagnostic cues based on the medical imaging dataset and the machine-readable representation of the at least one prior medical report.

6. The computer-implemented method of claim 5,
wherein the machine-readable representation of the at least one prior medical report classifies multiple sections of the at least one prior medical report, and
wherein different ones of the multiple sections are provided to different input channels of the processing algorithm.

7. The computer-implemented method of claim 1, further comprising:
obtaining at least one prior medical imaging dataset of at least one prior examination, and wherein
the determining determines the one or more diagnostic cues based on the medical imaging dataset and the at least one prior medical imaging dataset of the at least one prior examination.

8. The computer-implemented method of claim 1, further comprising:
obtaining at least one prior medical report of at least one prior examination of the patient, wherein
the processing algorithm is machine-learned and includes multiple encoder branches configured to determine respective latent features, and
different ones of the multiple encoder branches are associated with the at least one prior medical report and the medical imaging dataset of the current examination.

9. The computer-implemented method of claim 8,
wherein at least one encoder branch, of the multiple encoder branches, associated with the at least one prior medical report includes an attention layer for determining shortcuts between the respective latent features and a respective input to the processing algorithm.

10. The computer-implemented method of claim 8, further comprising:
merging, via the processing algorithm, the respective latent features to obtain merged latent features, wherein
the processing algorithm includes a decoder branch for reconstructing the one or more diagnostic cues based on the merged latent features.

11. The computer-implemented method of claim 8, further comprising:
obtaining multiple prior medical reports of multiple prior examinations of the patient; and wherein
the processing algorithm includes a multiple-instance pooling layer to merge latent features obtained from a respective encoder branch, of the multiple encoder branches, used to encode each one of the multiple prior medical reports.

12. The computer-implemented method of claim 11,
wherein the multiple-instance pooling layer comprises attention weighting for the multiple prior medical reports.

13. The computer-implemented method of claim 1,
wherein the determining determines multiple diagnostic cues sequentially,
wherein the computer-implemented method further includes obtaining user feedback associated with the multiple diagnostic cues, and
wherein said controlling of the user interface, said determining of the multiple diagnostic cues, and said obtaining of the user feedback is implemented in an entangled manner, so that a subsequent diagnostic cue of the multiple diagnostic cues depends on the user feedback associated with a preceding diagnostic cue among the multiple diagnostic cues.

14. A device comprising:
at least one processer configured to execute computer readable instructions to cause the device to
obtain a medical imaging dataset of a current examination of a patient,
determine, based on the medical imaging dataset, one or more diagnostic cues using a processing algorithm, the one or more diagnostic cues being associated with patient-specific diagnostic findings, and
control a user interface to pose the one or more diagnostic cues to a user, as part of a workflow for drawing up a current medical report for the current examination.

15. A non-transitory computer readable medium storing program code that, when executed by at least one processor, causes the processor to:
obtain a medical imaging dataset of a current examination of a patient,
determine, based on the medical imaging dataset, one or more diagnostic cues using a processing algorithm, the one or more diagnostic cues being associated with patient-specific diagnostic findings, and
control a user interface to pose the one or more diagnostic cues to a user, as part of a workflow for drawing up a current medical report for the current examination.

16. The computer-implemented method of claim 4, wherein the determining determines the one or more diagnostic cues based on the medical imaging dataset and the current medical report.

17. The computer-implemented method of claim 6, wherein the different input channels are associated with different encoder branches of the processing algorithm.

18. The computer-implemented method of claim 2, further comprising:
obtaining at least one prior medical report of at least one prior examination of the patient; and
analyzing, using a natural language processing algorithm, the at least one prior medical report to obtain a machine-readable representation of the at least one prior medical report, wherein
the determining determines the one or more diagnostic cues based on the medical imaging dataset and the machine-readable representation of the at least one prior medical report.

19. The computer-implemented method of claim 5, further comprising:
obtaining at least one prior medical report of at least one prior examination of the patient, wherein
the processing algorithm is machine-learned and includes multiple encoder branches configured to determine respective latent features, and
different ones of the multiple encoder branches are associated with the at least one prior medical report and the medical imaging dataset of the current examination.

20. The computer-implemented method of claim 9, further comprising:
merging, via the processing algorithm, the respective latent features of the multiple encoder branches to obtain merged latent features, wherein
the processing algorithm includes a decoder branch for reconstructing the one or more diagnostic cues based on the merged latent features.
